(19) United States
(12) Patent Application Publication
(10) Pub. No.: US 2004/0058453 A1
(43) Pub. Date: Mar. 25, 2004

(54) Reaction pouch comprising an analytical sensor

(75) Inventors: M. Benton Free, St. Paul, MN (US); Del R. Lawson, Cottage Grove, MN (US); Lester H. McIntosh III, Green Lane, PA (US); Stephen B. Roscoe, St. Paul, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/251,066

(22) Filed: Sep. 20, 2002

(51) Int. Cl. G01N 33/00

(52) U.S. Cl. 436/183; 422/50; 422/61; 422/68.1; 422/99

(57) ABSTRACT

A reaction device comprises a flexible, fluid-impervious pouch and an analytical sensor for real time, in situ, reversible measurement of properties of materials within the pouch. The sensor can be integrally connected to the pouch or it can be free of such connection. Preferably, the analytical sensor comprises a responsive element that can be located inside or on the pouch, a processing element that can be located outside the pouch, and a means for transmitting information between the responsive element and the processing element. The transmitting means can include one or more of electrical, optical, magnetic, nuclear and mechanical means. The pouch can be used singly or it can be a member of a combinatorial array of pouches that can be used in producing a library of materials. A method monitors changes in properties of materials within the pouch.

### FIELD OF THE INVENTION

A reaction device includes a flexible pouch comprising an analytical sensor. A method monitors changes in properties of materials within the pouch. The reaction device and method are useful in providing and monitoring chemical, physical, and biological syntheses.

### BACKGROUND OF THE INVENTION

Rapid screening of results of a physical or chemical reaction is a desirable feature when using a single reaction vessel or when using a multiplicity of reaction vessels for producing a combinatorial array of members of a library. Real time screening, i.e., accessing changes in properties of materials within a reaction vessel as they occur, is the ultimate goal.

One-dimensional arrays of chemical compounds are known (WO 99/42605) in which the compounds are synthesized on an elongated support (string) and the frequency with which each component appears is used for identification. WO 99/32705 describes a string of pouches, each of which is intended to contain a different compound. The pouches are composed of microfilamentous polypropylene to allow the permeation of fluids, and are also radiation treated so that the library elements can be attached to the pouch surface.

A particularly attractive way to synthesize an array of chemical compositions is to prepackage monomeric chemical species into a sealed package along with appropriate photopolymerization initiators and then to polymerize the monomers. For example, U.S. Pat. No. 5,804,610 teaches methods for preparing viscoelastic compositions (e.g., adhesives such as hot melt adhesives) in which a pre-viscoelastic composition is combined with a packaging material and then polymerized by transmissive energy. However, the determination of the chemical and physical properties of the polymerized compositions is achieved after the syntheses are completed.

It is known to use sensors to determine the properties of a material in a sealed, pouch-like system. For example, PCT International Publication No. WO/00/10504 describes the use of a microporous membrane sensor that may be attached to the wall of a blood storage container. The membrane includes pores that can be filled with an erodible substance responsive to a change in pH within the container. If the pH of the liquid in the pouch drops significantly, the substance in the pores erodes, enlarging the pores and allowing a portion of the blood product to pass through the pores into a contained space where it can be visibly detected. PCT International Publication No. WO/92/19764 relates to a growth monitoring apparatus for collected transfusable bodily fluids. In particular, the apparatus involves a flexible blood collection bag or a sample bag containing microbial growth media. A sensor attached to the inside wall of the bag is used to noninvasively detect microbial contamination within the bag. This invention also relates to a method to detect microbial growth in a blood collection bag immediately prior to transfusion. The sensor is used to monitor bacterial growth in a collection bag of bodily fluid that is transfused into a patient by externally monitoring the emitted fluorescence of the fluorescence based dye.

### SUMMARY OF THE INVENTION

Briefly, the present invention provides a reaction device comprising a flexible, fluid-impervious pouch, the reaction device further comprising an analytical sensor for real time, in situ, reversible measurement of properties of materials within the pouch. The sensor can be integrally connected to the pouch or it can be free of such connection. Preferably, the analytical sensor comprises a responsive element which can be located inside or on the pouch, a processing element which can be located outside the pouch, and a means for transmitting information between the responsive element and the processing element. The transmitting means can be a mechanical element, for example, a wire or a fiber optic cable, or it can be a radiation element such as acoustic waves, actinic radiation, nuclear radiation, or magnetism. The pouch can be used singly or it can be a member of a combinatorial array of pouches that can be used in producing a library of materials. Preferably, the pouch is self-supported. The reaction device is particularly useful for real time, continuous measurements of properties of components, intermediates, and products in chemical, physical, and biological syntheses, blends, and formulations.

More particularly, each reaction device comprises a reaction pouch, and the reaction device also comprises one or more analytical sensors for monitoring changes, for example, in physical or chemical properties of materials as the changes occur at one or more locations within the pouch. It is understood that monitoring of reversible biological properties that are chemical or physical in nature, such as color, turbidity, fluorescence, etc., are included within the scope of this invention. The analytical sensor comprises a responsive element inside or on the pouch that converts chemical, physical, or biological information (output) into electrical or electromagnetic signals, and a processing element that converts electrical or electromagnetic signals into usable information. The responsive element as well as a transmitting means can be located inside the pouch (free floating or attached) or it can be on the inside wall of the pouch or it can be a portion or all of the wall of the pouch. Alternatively, the responsive element can be sealed in an edge of the pouch. The responsive element can be addressed by one or more external electrodes or it can be addressed remotely. Preferably, the responsive element comprises one or more of the following properties: flexibility, reusability, non-degradability, disposability, and low-cost.

In another aspect, the present invention provides a method for real time, in situ, reversible monitoring of changes in material properties of contents of a pouch comprised in a reaction device, the method comprising the steps of:

a) providing a reaction device comprising a flexible, sealed, fluid-impervious pouch including one or more reaction components, the reaction device also comprising an analytical sensor operating under a measurement protocol for real time, in situ, reversible measurement of properties of materials within the pouch, the analytical sensor comprising a responsive element inside or on the pouch for converting chemical or physical information (output) into electrical or electromagnetic signals, a processing element for converting electrical or electromagnetic signals into usable information, and a means for transmitting information between the responsive element and the processing elements,

b) exposing the pouch to a controlled environment to cause the reaction components to interact to form one or more of blends, reaction products, and formulations, and

c) causing the responsive element and the processing element of the sensor to monitor changes in material properties occurring within the pouch, and

d) optionally, using the processing information to modify one or more of the components, controlled environment, and the measurement protocol.

The analytical sensors can be used to monitor one or a plurality of physical, chemical, and biological properties of reaction components, intermediates, and products within the pouch.

The reaction device of the present invention possesses superior properties compared with conventional reaction vessels and provides the following advantages: it allows for rapid evaluation of pouch reactants, intermediates and products without time-consuming extraction and purification procedures; it makes possible the monitoring of reaction progress in situ, in real time, thereby facilitating reaction optimization as, for example, in monitoring the degree of cure of a polymer; it enables study of fundamental reaction kinetics under process conditions; it allows for monitoring of reversible properties, such as temperature, crystal frequency, etc., and it can allow for in-process evaluation to ensure, for example, that a manufacturing process stays under control.

In this application:

“actinic radiation” means electromagnetic radiation, preferably UV (ultraviolet), microwave, and IR (infrared);

“film” means a sheet-like material suitable for making into a pouch;

“flexible” means can be bent around a rod of diameter 10 cm, preferable 1 cm, more preferable 1 or 2 mm, and most preferably 0.25 mm or less;

“free-floating” means having the freedom to move in at least one direction;

“in situ” measurement of properties means the responsive element is in physical contact with the contents of the pouch;

“pouch” means a flexible, sealed or unsealed, preferably self-supported bag, package, or reaction vessel made of a film that preferably is inert to materials within it and, when sealed, is impervious to fluid in the surrounding environment; preferably it is of unitary construction although a combination of compatible materials can be used;

“real time” means a measurement that is performed essentially simultaneously with the event itself;

“reversible” means capable of measuring both the increases and the decreases in the values of properties of the materials of the pouch in real time; and

“unitary construction” means of one material, except where a septum is present, the septum can be of a different material.

### BRIEF DESCRIPTION OF THE DRAWING

FIG. 1a shows a perspective view of one embodiment of a reaction device of the invention including a three component analytical sensor having a responsive element sealed in one end of a pouch, a processing element for converting signals into useful information, and a physical transmitting means for transmitting information between the responsive element and the processing element.

FIG. 1b shows a perspective view of one embodiment of a reaction device of the invention including an analytical sensor comprising a remotely addressed responsive element sealed in one end of a pouch.

FIG. 2a shows a perspective view of one embodiment of a reaction device of the invention including a three component analytical sensor having one end of a responsive element attached to the inside of the pouch.

FIG. 2b shows a perspective view of one embodiment of a reaction device of the invention including an analytical sensor comprising a remotely addressed responsive element free-floating inside the pouch.

FIG. 3a shows a perspective view of one embodiment of a reaction device of the invention including a three component analytical sensor having a responsive element attached to the inside of a wall of a pouch.

FIG. 3b shows a perspective view of one embodiment of a reaction device of the invention including an analytical sensor comprising a remotely addressed responsive element attached to the inside of a wall of a pouch.

FIG. 4a shows a perspective view of one embodiment of a reaction device of the invention including a three component analytical sensor having a responsive element incorporated in the body of a pouch (akin to a patch in the body of the pouch).

FIG. 4a′ shows a cross-sectional view of FIG. 4a taken along line 4a′-4a′.

FIG. 4b shows a perspective view of one embodiment of a reaction device of the invention including an analytical sensor comprising a remotely addressed responsive element incorporated in the body of a pouch (akin to a patch in the body of the pouch).

### DETAILED DESCRIPTION OF PREFERRED EMBODIMENTS

The present invention provides for in situ and real-time sensing of reaction, intermediate, and product properties using a flexible pouch or an array of pouches as the reaction vessel(s). The present invention incorporates analytical sensors, responsive to material properties of interest, into each pouch.

These material properties can be measured with a number of different sensors including but not limited to: thermocouples, interdigitated transducers (IDTs), acoustic sensors including surface acoustical wave devices (SAWs) and quartz crystal microbalances (QCMs). These devices are known and accessible and are capable of responding to a wide range of material properties such as mass, density, modulus, electrical conductivity, pH, etc.

Of particular interest is an inexpensive, disposable sensor having a responsive element that can be made by depositing a copper circuit pattern on polymer film (Microflex™, 3M Company, St. Paul, Minn.). For methods and materials useful for producing these sensors, see U.S. Pat. Nos., 5,227,008, 6,071,597, and 6,177,357, which patents are incorporated herein by reference.

U.S. Pat. No. 5,227,008 relates to a process for making flexible circuits wherein the etching of a polymeric film is accomplished by dissolving portions thereof with concentrated aqueous base using an aqueous processible crosslinked photoresist as a mask, comprising the steps of laminating the resist, developing the resist with a dilute aqueous solution until desired image is obtained, etching portions of the polymeric film not covered by the crosslinked resist with a concentrated base at a temperature of from about 50° C. to about 120° C., and then stripping the resist off the polymeric film.

U.S. Pat. No. 6,071,597 relates to a flexible printed circuit comprising:

a) at least one layer of polymer dielectric material, b) at least one layer of electrically conductive material thereover, and c) at least one circuit trace, each of said dielectric layers and each of said conductive layers having two major surfaces, at least one layer selected from a dielectric layer or a conductive layer having at least one aperture therein, wherein at least one of said dielectric layers has a material selected from the group consisting of diamond-like carbon, hydrogenated diamond-like carbon, functionalized diamond-like carbon, silicone nitride, boron nitride, silicon carbide, silicon dioxide and boron trifluoride coated on at least a portion of at least one major surface of said dielectric layers, said material having a Young's Modulus of from about 100 to about 200 Gpa, a dielectric constant between 45 MHz and 20 GHz of from about 8 to about 12, and a Vickers hardness of from about 20000 to about 9000 kg/mm².

U.S. Pat. No. 6,177,357 relates to a process for making a flexible printed circuit wherein etching of a polymeric film is accomplished by dissolving portions thereof with concentrated aqueous base, using a UV-curable, 100% active liquid photoresist as a mask, comprising the steps of a) laminating said resist on a flexible substrate comprising a layer of polymer film and a thin layer of copper, b) exposing at least a portion of said resist thereby crosslinking said exposed portions, c) plating circuitry atop said thin copper layer to desired thickness, d) etching portions of said polymeric film not covered by the crosslinked resist with a concentrated base at a temperature of from about 70° C. to about 120° C., e) stripping said resist off said polymeric film with dilute basic solution, and f) etching said thin copper layer to obtain circuitry.

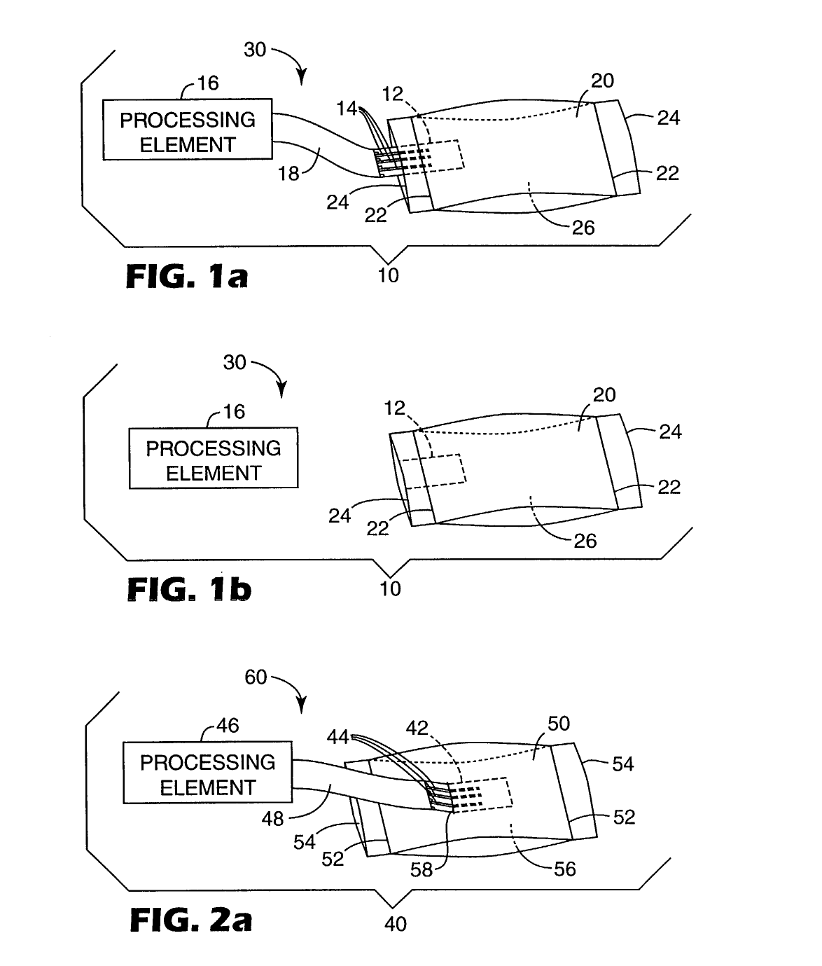

The reaction devices and method of the invention are applicable to both actinic radiation cured, preferably UV cured, and thermally cured polymerizations. In one embodiment, an array of connectors can be sealed through one end of the pouch, with an IDT in direct contact with the contents of the pouch. Connectors can be externally located, as shown in FIGS. 1a, 2a and 3a. of the Drawing. This embodiment is well-suited, for example, to UV-initiated polymerizations where it is possible to follow the progress of a reaction as, for example, in a UV-cured polymerization reaction where the monomers are expected to have a significant absorption cross-section at the excitation wavelength. Those in thicker parts of the pouch will be exposed to less UV light and will cure more slowly. This is particularly noticeable with UV-cured acrylates where polymer near the edges of the pouch can be stiffer than that in the center.

In another embodiment, where, for example, water can be used as a temperature-control medium, a radio frequency antenna can be incorporated into the detecting element so that the sensor need not penetrate the pouch. Such an antenna can be addressed remotely, providing benefits not only in situations where sealing is an issue, but also where it is difficult, or time-consuming, to attach external connectors to the pouch to be evaluated as is shown in FIGS. 1b, 2b and 3b.

Pouches are useful in the present invention singly or in a combinatorial array and have been described in assignee's copending patent application U.S. Ser. No. 09/793,666 (Attorney's Docket No. 55970US002), filed Feb. 22, 2001, which is incorporated herein by reference.

More particularly, forming a flexible pouch can be accomplished in various ways, for example, by heat sealing two lengths of a thermoplastic film together across the bottom and on each lateral edge on a device such as a liquid form-fill-seal machine (for example, using Model 70A2C from General Packaging, Houston Tex.) or manually to form an open ended pouch. Also, a single length of film can be folded and sealed on two edges, charged with components and the remaining edge sealed. Alternatively, a tube of film can be sealed at one end, charged with components and sealed at the opposite end. Pouches can be of any shape that is useful but pouches having rectangular or square surfaces are preferred. With certain sensors it may be desirable to use a curable adhesive to seal the pouch around the connector or the sensor.

Generally, after the components are introduced into a pouch, it is heat sealed to completely surround the components. The sealing temperature is generally above the softening point and below the melting point of the film used to form the pouch. Removal of most of the air from the pouch prior to sealing is preferred. This may be done by, for example, evacuation or mechanical compression. Seals can be affected in any of a number of different configurations to form multiple pouches across and down the length of the film. For example, in addition to seals on the lateral edges, a seal can also be formed down the center of the film, which, upon sealing of the top and bottom edges, will form two packages. The packages can be left attached to each other by the center seal or cut into individual pouches. In another embodiment, one or a plurality of pouches, herein referred to as captive pouches, can be included inside the original pouch in order to add additional components. This can be accomplished either by pre-sealing the additional components into one or more smaller separate captive pouches which can be included during the charging of the initial components or they can be incorporated as smaller internal pouches inside the original pouch. The captive pouches can be free floating or they can be presealed into one or more edges of the primary pouch. The captive pouches containing additional components can be made of material that allows rupture more easily than the primary pouch, effecting contact of the additional components with the primary components. Forming the captive pouches of thinner material than the primary pouch or by utilizing a laminated pouch with a lower melting point facilitates rupturing of the captive pouches. In the former case, the captive pouches can then be ruptured by mechanical agitation such as kneading or compression. In the latter case, an elevated temperature preferably coupled with mechanical agitation can cause rupture of the captive pouches. In an alternative embodiment, captive pouches can be made of a material that decomposes under actinic energy (or other types of energy), which causes the pouch to rupture and release its contents. In another embodiment, the primary pouch can be fitted with a septum inlet to allow resealable entry into the pouch for charging additional components, without disturbing the integrity of the pouch for storage.

Pouches preferably comprise a flexible film, which can be UV or IR transparent in certain embodiments. Thermoplastic films are available from many commercial sources, for example, Huntsman Packaging, Rockford Ill. The specific thermoplastic film utilized will depend to a large extent on the composition and melting point of the components and products contained within the pouch, with the softening point of the film generally being less than 125° C. Single layer or multi-layer laminated pouches can be made of flexible thermoplastic polymeric film such as homo- and copolymers of polyolefins, polydienes, polystyrenes, polyesters, polyethers, halogenated polyolefins, polyvinylalcohol, polyamides, polyimines, polycycloolefins, polyphosphazines, polyacetates and polyacrylates. Preferred thermoplastic film materials include low density polyethylene (LDPE), linear low density polyethylene (LLDPE), polypropylene (PP), polyethyleneterephthalate (PET), polytetrafluoroethylene (PTFE), polyvinylidenefluoride (PVF), polyvinylacetate (PVA), copolymers of ethylene and vinyl acetate, vinylidene fluoride, vinyl chloride, teterafluoroethylene and propylene. Sheets of film are commercially available as noted above, and they can be useful in producing packaged members. Such pouches that can be used singly or in the combinatorial libraries of the present invention are disclosed for example, in U.S. Pat. No. 5,902,654, incorporated herein by reference for this purpose. Methods for preparing viscoelastic compositions (e.g., adhesives such as hot melt adhesives) in which a pre-viscoelastic composition (e.g., a pre-adhesive composition) is combined with a packaging material and then polymerized by transmissive energy are disclosed in U.S. Pat. Nos. 5,804,610 and 5,932,298, which are incorporated herein by reference for these methods and compositions. A process that involves the packaged polymerization of olefinic monomer(s) and catalyst systems comprising a transition metal species that mediates the polymerization of the monomer(s) is disclosed in U.S. Pat. No. 5,902,654, which is incorporated herein by reference for the process and compositions. This process provides a way to use the resultant polymer without extensive further processing. Other films that can be useful in the present invention include metal films, for example, foils of copper and aluminum and any metal in Groups 2, 3, 4, 5, 6, 7 and 8 on the Periodic Table, as well as composite materials that combine polymer films, metal foils, paper materials, and woven and nonwoven textile materials such as cotton, wool, fiberglass, and polymer fibers.

The thickness of the film utilized for the primary pouch generally varies between about 5 μm -3 mm, preferably 25-250 μm, more preferably 50-150 μm. The thickness of the film also varies depending on the temperature or conditions to which the components of the pouch are to be subjected, with thicker films utilized for high and low temperature applications or applications requiring mechanical manipulation. Captive pouches can be formed of the same or different material and can be the same thickness as the primary pouch or they can be thinner, preferably between about 1 μm -1 mm, more preferably 5-150 μm, most preferably between 15-50 μm. The size of the pouch can be of any desired dimensions.

However, persons skilled in the art will recognize that the dimensions of the pouch enable control of the reaction conditions within the pouch to be accomplished. For example, bulk reactions, due to their concentrated mass, require pouches of smaller dimensions than do solution or suspension reactions. This is due to the higher concentration of reacting species and the need for larger surface area to remove thermal energy generated during typical chemical reactions. Solution and suspension reactions on the other hand contain lower concentrations of reacting species and as such require less surface area for thermal energy removal. Primary pouch dimensions for bulk reactions can be of varying sizes, but are generally less than about 100 cm×100 cm, preferably less than about 20 cm×20 cm, more preferably about 13 cm×7 cm or even 2 cm×1 cm or less. The size of the captive pouches adheres to the same constraints and may be of any size provided that it fits within the primary pouch. One skilled in the art will recognize that the type of additional component(s) added from the captive pouches may dictate the size of the primary pouches. For example, if an additional component is a catalyst, the size of the captive pouch required may be quite small in size, e.g., 1 cm×1 cm, whereas if the captive pouch contains a comonomer for a solution copolymerization, the captive pouch may be quite large, e.g., for example, 50 cm×50 cm or less, preferably 10 cm×10 cm or less, most preferably from about 4 cm×5 cm to about 5 mm×5 mm.

Pouches containing components can be used singly or they can be linearly and/or horizontally attached to each other or physically separated from each other. After sealing, they can be conveyed through a reaction zone, which can subject each pouch to the same or differing reaction conditions and dwell times. This substantially increases the scope and number of reactions that can be encompassed in an individual library. The reaction zone can be as simple as a constant temperature water bath or as elaborate as a controlled temperature ultrasonic bath. Typically, the duration of reaction time for each pouch can be controlled by the length of the reaction zone utilized. Longer reaction times can require longer reaction zones. Mixing of the components within the pouches can be effected by, but is not limited to, mechanical agitation, e.g., kneading rollers, or controlled pressure gradient changes within a sealed bath, or ultrasonic agitation.

More specifically, the reaction zone can be a liquid, gaseous or solid bath used to initiate and promote chemical or physical reactions and/or control temperature. Formation of the library arrays of the invention, as by chemical or physical reactions, can be facilitated by a variety of energy means, including but not limited to actinic radiation, including thermal, mechanical or ultrasonic energy. Examples of reaction zone baths include but are not limited to water baths, convection ovens, salt baths, and fluidized beds. After passage through the reaction zone, the pouches optionally can be separated and subject to various evaluations or stored for later evaluation and analysis.

In one embodiment of the invention, the separate, self-supported pouches can be placed into and removed manually from one or more reaction zones. In this embodiment, while the process is not mechanically continuous, the products obtained can be subjected to the same constraints as in the following embodiments in that individual pouches can be subject to differing reaction zone conditions and dwell times.

In a preferred alternative embodiment the primary pouches can be separate, freestanding, self-supported entities which are temporally spaced with respect to each other. They can be supported by or fastened individually, for example, by means of pins or clamps to a conveyance apparatus such as a moving belt or track for transportation through a reaction zone. This can be a continuous process, wherein, by changing the conditions of the reaction zone (for example temperature, radiant energy, mechanical energy, ultrasonic energy, etc.) and by varying the time spent in the reaction zone, reaction conditions can be varied with each individual pouch, if so desired.

In a most preferred embodiment, the pouches can be joined to each other at one or more edges linearly and/or horizontally. As mentioned above they can be supported by or fastened to a conveyance apparatus. In this embodiment, the pouches are also temporally spaced with respect to each other and can be transported through the reaction zone by various means including rollers, belts, or by rolling onto a spool. Once again, this can also be a continuous process wherein the conditions and duration of time spent within the reaction zone can be varied for each individual pouch if so desired.

An analytical sensor useful in the present invention includes any measurement device that provides analytical information reflecting chemical, physical, or biological properties of the sample being investigated. Examples of mechanical properties that can be measured include but are not limited to: density; strain; force; torque; pressure; viscosity; surface tension; temperature; heat flux; capacitance; permittivity; complex impedance; color; refractive index (RI); wavelength; thermal conductivity; and rheologic and morphologic properties.

Examples of chemical properties that can be measured include but are not limited to: concentration; reaction rate; binding constant; presence/absence of a species; identity of a species; quantification of reactants, intermediates and products; molecular weight; polydispersity; pH; and moisture content.

Many responsive elements can be useful in the present invention and include, for example but are not limited to: piezoelectric devices; electrochemical devices; optical probes; calorimetric devices, thermistors/thermocouples; inter-digitated transducers; resistance devices; hall resistance (magnetic capabilities) devices; thermal conductivity devices ( e.g. one heater and one temperature sensor in proximity); and cantilever probes. A wide variety of transmitting means are available and include, for example, electrical, optical, magnetic, nuclear, and mechanical means. More specifically, wires, fiber optical cable, radio frequency identification (RFID), acoustic waves, actinic radiation, nuclear radiation, and magnetism can be used to transmit information between responsive and processing elements.

FIG. 1a is a perspective view of reaction device 10 of the present invention comprising flexible pouch 20 and analytical sensor 30. Analytical sensor 30 comprises responsive element 12, processing element 16, and transmitting means 18 for physically transmitting information (e.g., by wire, fiber optical cable, etc.) between responsive element 12 and processing element 16. Pouch 20 comprises seals 22 at or near its ends 24. Prior to use of pouch 20, one or both of seals 22 can be open for loading of materials. Connectors 14 of transmitting means 18 can be located outside pouch 20. Connectors 14 communicate with processing element 16 by any suitable means for transmitting information between responsive element 12 and processing element 16. Responsive element 12 can be attached to pouch 20 only at seal 22 so as to be free-floating in pouch 20 or it can also be attached to inside wall 26 of pouch 20 at one or more points (not shown), in which case it may be partially or totally anchored inside pouch 20.

FIG. 1b is a perspective view of reaction device 10 of the present invention comprising flexible pouch 20 and analytical sensor 30. Analytical sensor 30 comprises responsive element 12, processing element 16, and a transmitting means (not shown) for transmitting information between responsive element 12 and processing element 16. Pouch 20 comprises seals 22 at or near its ends 24. Prior to use of pouch 20, one or both of seals 22 can be open for loading of materials into pouch 20. Responsive element 12 can be attached to pouch 20 only at seal 22 near end of pouch 24 so as to be free-floating in pouch 20 or it can also be attached to inside wall 26 of pouch 20 at one or more points, in which case it may be partially or totally anchored in pouch 20. Responsive element 12 can be addressed remotely by processing element 16. Transmitting means (not shown) for remotely transmitting information from responsive element 12 to processing element 16 can include many known forms of energy, for example, acoustic waves, actinic radiation, nuclear radiation, and magnetism.

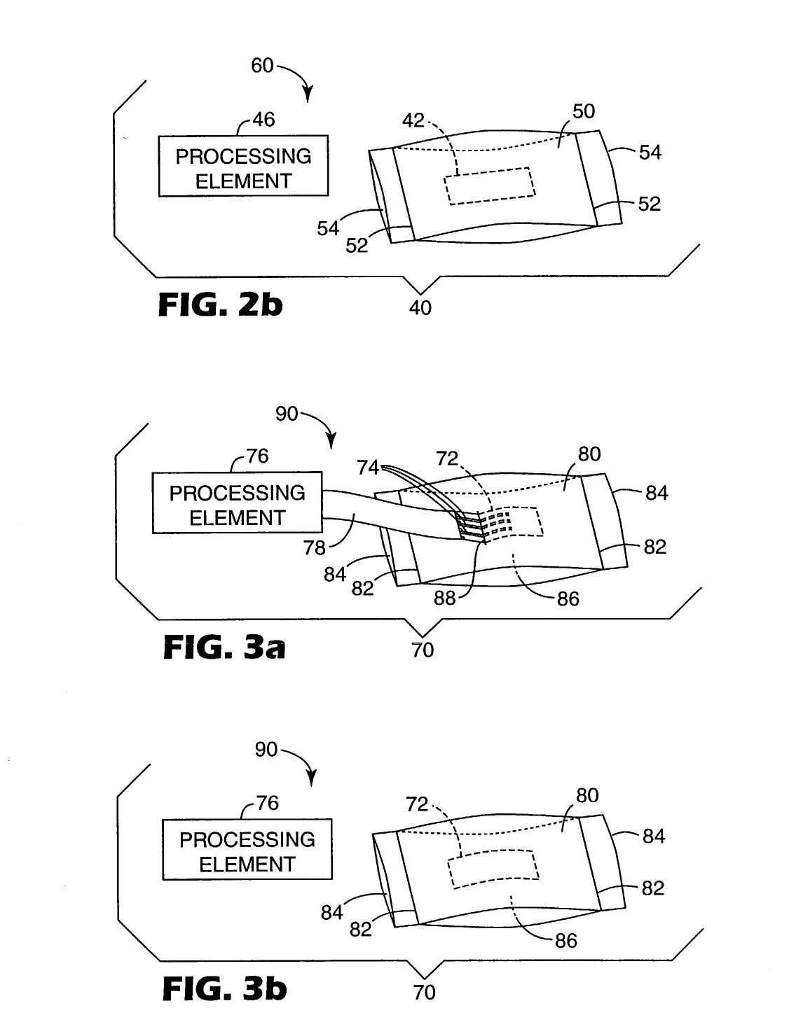

FIG. 2a is a perspective view of reaction device 40 of the present invention comprising flexible pouch 50 and analytical sensor 60. Analytical sensor 60 comprises responsive element 42, processing element 46, and transmitting means 48 for transmitting information between responsive element 42 and processing element 46. Pouch 50 comprises seals 52 at or near its ends 54. Prior to use of pouch 50, one or both of seals 52 can be open for loading of materials into pouch 50. Connectors 44 of transmitting means 48 are located outside and, optionally, inside pouch 50 and pass through seal 58. Connectors 44 communicate with processing element 46 by any suitable means for transmitting information between responsive element 42 and processing element 46. Transmitting means 48 includes mechanical elements such as a wire or fiber optic cable. Responsive element 42 is attached to the body of pouch 50 at seal 58 and otherwise can be free-floating inside pouch 50 or it can also be attached to inside wall 56 of pouch 20 at one or more points (not shown).

FIG. 2b is a perspective view of reaction device 40 of the present invention comprising flexible pouch 50 and analytical sensor 60. Analytical sensor 60 comprises responsive element 42, processing element 46, and a transmitting means (not shown) for transmitting information between responsive element 42 and processing element 46. Pouch 50 comprises seals 52 at or near its ends 54. Prior to use of pouch 50, one or both of seals 52 can be open for loading of materials into pouch 50. Responsive element 42 is free-floating inside pouch 50. Responsive element 42 is addressed remotely by processing element 46. Transmitting means (not shown) for remotely transmitting information from responsive element 42 can include many known forms of energy, for example, acoustic waves, actinic radiation, nuclear radiation, and magnetism.

FIG. 3a is a perspective view of reaction device 70 of the present invention comprising flexible pouch 80 and analytical sensor 90. Analytical sensor 90 comprises responsive element 72, processing element 76, and transmitting means 78 for transmitting information between responsive element 72 and processing element 76. Pouch 80 comprises seals 82 at or near its ends 84. Prior to use of the pouch 80, one or both of seals 82 can be open for loading of materials into pouch 80. Processing element 72 can be attached to pouch 80 and connectors 74 at seal 88. Connectors 74 of transmitting means 78 are located outside, and optionally, inside pouch 80 and pass through seal 88. Connectors 74 communicate with processing element 76 by any suitable means for transmitting information between responsive element 72 and processing element 76. Transmitting means 78 includes mechanical elements such as a wire or fiber optic cable. Responsive element 72 is attached to pouch 80 at seal 88.

FIG. 3b is a perspective view of reaction device 70 of the present invention comprising flexible pouch 80 and analytical sensor 90. Analytical sensor 90 comprises responsive element 72, processing element 76, and a transmitting means (not shown) for transmitting information between responsive element 72 and processing element 76. Pouch 80 comprises seals 82 at or near its ends 84. Prior to use of the pouch 80, one or both of seals 82 can be open for loading of materials into pouch 80. Responsive element 72 is attached to inside wall 86 of pouch 80. Responsive element 72 is addressed remotely by processing element 76. Transmitting means (not shown) for remotely transmitting information from responsive element 72 to processing element 76 can include many known forms of energy, for example, acoustic waves, actinic radiation, nuclear radiation, and magnetism.

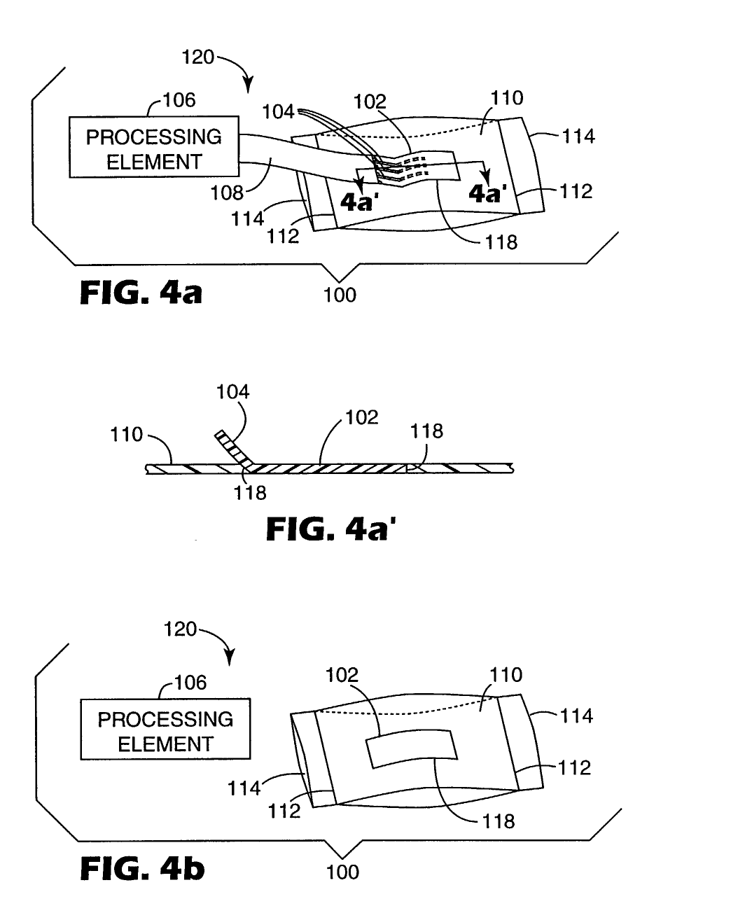

FIG. 4a is a perspective view of reaction device 100 of the present invention comprising flexible pouch 110 and analytical sensor 120. Analytical sensor 120 comprises responsive element 102, processing element 106, and transmitting means 108 for transmitting information between responsive element 102 and processing element 106. Pouch 110 comprises seals 112 at or near its ends 114. Prior to use of pouch 110, one or both of seals 112 can be open for loading of materials into pouch 110. Responsive element 102 is contiguous with, and forms a part of the body of pouch 110. Connectors 104 are located outside pouch 110 and optionally can be incorporated inside responsive element 102. Responsive element 102 is sealed in the body of pouch 110 around perimeter seal 118. Connectors 104 are attached to responsive element 102 at seal 118 and communicate with processing element 106 by any suitable means for transmitting information between responsive element 102 and processing element 106. Transmitting means 108 includes mechanical elements such as a wire or fiber optic cable.

FIG. 4a′ shows a cross-sectional view of FIG. 4a taken along line 4a′-4a and depicts pouch 110, responsive element 102 and its peripheral seal 118, and connectors 104 of responsive element 102.

FIG. 4b is a perspective view of reaction device 100 of the present invention comprising flexible pouch 110 and analytical sensor 120. Analytical sensor 120 comprises responsive element 102, processing element 106, and transmitting means (not shown) for transmitting information between responsive element 102 and processing element 106. Pouch 110 comprises seals 112 at or near its ends 114. Prior to use of pouch 110, one or both of seals 112 can be open for loading of materials into pouch 110. Responsive element 102 is contiguous with, and forms a part of, the body of pouch 110 at seal 118. Responsive element 102 is addressed remotely by processing element 106. Transmitting means (not shown) for remotely transmitting information from responsive element 102 to processing element 106 can include many known forms of energy, for example, acoustic waves, actinic radiation, nuclear radiation, and magnetism.

This invention finds utility in real time, in situ, reversible monitoring of chemical, physical, and biological properties of components, intermediates and products during the synthesizing, blending or formulating of organic, inorganic, and biological materials. It can be used, for example, in the creation of single species or libraries in organic synthesis, photochemistry, polymer synthesis, and synthesis of biological products. It can provide a linear and or horizontal array of library samples, preferably in quantities of 0.5 g up to and including commercially useful quantities, in flexible, impervious, sealable or sealed pouches.

The method is applicable to the large-scale production of commercial materials. The technique, therefore, can be used for manual creation of one pouch containing a formulation followed by a second pouch containing a different formulation and so on. It preferably can utilize an automated process in which filling of each pouch with reactants such as monomers, etc., can be varied using automatic dispensing systems and the pouches can be connected together. Such automatic methods for combining components are disclosed, for example, in U.S. Pat. No. 5,902,654, the methods being incorporated herein by reference.

Objects and advantages of the invention are further illustrated by the following examples, but the particular materials and amounts thereof recited in these examples, as well as other conditions and details, should not be construed to unduly limit this invention.

### EXAMPLES

This invention is further illustrated by the following examples, which are not intended to limit the scope of the invention. In the examples, all parts, ratios, and percentages are by weight unless otherwise indicated. All materials, unless otherwise stated, are available from the Aldrich Chemical Company, Milwaukee Wis.

Glossary

IDT—interdigitated transducer

M_{w}—weight average molecular weight

Mₙ—number average molecular weight

PD—polydispersity (=Mₙ/M_{w})

IBA—isobornyl acrylate

THFA—tertahydrofurfuryl acrylate

2-EHA—2-ethylhexyl acrylate

IOTG—isooctylthioglycolate

F—frequency

QCM—quartz crystal microbalance

T_{g}—glass transition temperature

D—electrical dissipation

### Example 1

Interdigitated transducers (IDTs), comprising 46 pairs of fingers at 75 μm pitch, were prepared by depositing 18 μm thick copper over a 1.5 μm nickel tie layer on 0.05 mm thick polyimide film and then covering with a thin layer of gold (0.75 μm) in accordance with U.S. Pat. No. 5,227,008. These flexible sensors were inserted into one end of a 10 cm section of polyethylene bag tubing (4.5 cm diameter×0.15 mm thick, catalog number 2062T23, McMaster Carr, Chicago Ill.) with the IDTs pointing inwards and electrodes for attachment extending outward of the tube (see FIG. 1a). The tubing was then thermally sealed across the lower part of the electrode with a thermally curable adhesive comprising an epoxidized styrene-butadiene-styrene block copolymer as exemplified in U.S. Pat. No. 6,294,270), simultaneously forming the lower edge of the pouch and embedding the responsive element within the pouch. The pouches were then filled with IBA, THFA, and 2-EHA in various ratios, as in Table 1 below. An ultraviolet (UV) initiator (Darocur 1173, Ciba Specialty Chemicals, Tarrytown N.Y.) and a charge transfer agent (isooctylthioglycolate Hampshire Chemicals, Lexington, Mass.) were added at 0.8 vol % and 0.25 vol %, respectively. The open pouches were then degassed with flowing N₂ for 5 min and then the remaining open end was heat-sealed.

The pouches were immersed in ice water and then exposed to UV light (350 nm Blacklight, Osram Sylvania, Danvers Mass.) at a distance of ˜10 cm for 2 hours 50 minutes to ensure complete polymerization. The electrodes of the IDTs were subjected to an AC potential, the frequency (F) of which was increased from 30 Hz to 1 MHz during the course of the measurement. The dissipation (D) of the resultant signal was monitored and recorded as a function of input frequency F. For each sample the dissipation increased, reached a maximum, and then subsided as the frequency increased. The frequency at which this maximum dissipation occurred (Fₘₐₓ) varied markedly for each sample. For comparison, the glass transition temperature (T_{g}) of each sample was assessed ex situ by removing a small portion of the contents of the pouch and recording a differential scanning calorimetry (DSC) trace at 10° C./min. Table 1 reports properties for representative samples taken from each pouch.

TABLE 1
Monitoring Data for Polymerization

| Components | Sample A | Sample B | Sample C | Sample D |
|---|---|---|---|---|
| 2-EHA | 4 mL | 5 mL | 6 mL | 2 mL |
| IBA | 4 mL | 2 mL | 6 mL | 5 mL |
| THFA | 4 mL | 5 mL | 0 mL | 5 mL |
| Darocur 1173 | 0.1 mL | 0.1 mL | 0.1 mL | 0.1 mL |
| IOTG | 0.03 mL | 0.03 mL | 0.03 mL | 0.03 mL |
| Properties | | | | |
| Fₘₐₓ (Hz) | 10000 | 250000 | 2000 | 150 |
| log (Fₘₐₓ) | 4 | 5.39 | 3.30 | 2.17 |
| T_{g} (° C.) | −10.2 | −26.9 | −8.9 | 3.9 |

The inverse relationship between T_{g} and log (Fₘₐₓ) demonstrates the ability to approximate thermochemical data from dissipation measurements. The data show the capacity of the present invention to evaluate useful chemical and physical properties of different copolymers.

### Example 2

An open polyethylene pouch containing interdigitated electrodes was prepared as in Example 1, filled with 4 mL each of IBA, 2-EHA and THFA and 0.04 vol % of Darocur 1173 and 0.025 vol % IOTG and then heated sealed. The monomer mixture was then irradiated with a 365 nm 8W UV source (UVP, Upland Calif.) at room temperature (25° C.) for 22 minutes. At various time intervals, the frequency dependant capacitance was measured by applying an AC potential to the IDT electrodes. The value of capacitance at 28 KHz for different cure times is reported in Table 2. The data of Table 2 show the change in capacitance of the sample with increasing reaction time. Decreased capacitance is related to increased polymer cure, increased molecular weight, increased viscosity, and completeness of cure.

TABLE 2
Time vs Capacitance

| time (sec) | Capacitance (pF) at 28 kHz |
|---|---|
| 0 | 32.78 |
| 40 | 33.01 |
| 80 | 32.91 |
| 140 | 31.88 |
| 200 | 31.26 |
| 283 | 29.84 |
| 343 | 27.50 |
| 403 | 25.40 |
| 463 | 25.32 |
| 583 | 25.13 |
| 703 | 24.67 |
| 823 | 24.23 |
| 1003 | 24.25 |
| 1183 | 24.21 |
| 1363 | 24.08 |
| 1543 | 24.12 |
| 1723 | 24.11 |
| 1903 | 24.03 |
| 2103 | 24.12 |
| 3423 | 23.92 |

### Example 3

Twenty-four IDT's, arranged in an array of six columns (labeled a-f) of four rows (labeled 1-4), were deposited on a sheet of polyimide as described in Example 1. The polyimide sheet comprising the array of sensors was then heat-sealed with the same thermally curable adhesive as in Example 1 to join with a sheet of polyethylene approximately 4.5 cm×4.5 cm×0.15 mm thick (prepared from the polyethylene bag tubing of Example 1 ), so that the IDTs faced the polyethylene. Three edges were sealed first to form an open-ended pouch that was then filled with 4 mL each of IBA, THFA, and 2-EHA. Initiator (Dacor 1173) and charge transfer agent (IOTG) were added at 0.4 vol % each. The open pouch was then degassed with flowing N₂ for 5 min and then the open end of the pouch was hot-sealed.

The pouch was immersed in ice water and exposed to UV light (350 nm Blacklight, Osarm Sylvania Mass.) at a distance of ˜10 cm for 5 minutes to effect

The electrical properties were measured for each of the IDTs as described in Example 1, and the results are reported in Table 3, below. Note that position 3b did not yield a signal.

TABLE 3
Capacitance (pF) at different locations within the pouch

| | a | b | c | d | e | f |
|---|---|---|---|---|---|---|
| 1 | 21.16 | 21.6 | 21.61 | 22.06 | 22.26 | 23.62 |
| 2 | 21.32 | 21.89 | 21.79 | 22.59 | 23.03 | 24.54 |
| 3 | 21.34 | | 22.01 | 22.68 | 23.37 | 25.04 |
| 4 | 21.73 | 21.98 | 22.31 | 22.62 | 23.19 | 24.79 |

The data of Table 3 show the variation in the value of capacitance at 28 kHz for different spatial positions within the pouch. This example illustrates the unique understanding of the spectral distribution of properties that were captured using an array of sensors within a reaction vessel.

### Example 4

Into one of the open ends of a 10 cm piece of polyethylene bag tubing (4.5 cm diameter×0.5 mm thick), as in Example 1, was placed a TEFLON (polytetrafluoroethylene) coated k type thermocouple (Omega Engineering, Stamford Conn.). The end of the tubing was then heat sealed and the opening around the thermocouple was sealed using a small amount of 5 minute epoxy (Devcon, Danvers Mass.). The pouch was placed in a dry box (Vacuum Atmospheres, Hawthorne, Calif.) and the thermocouple was attached to an 871A digital thermometer (Omega Engineering). Through the open end of the pouch was then added 5.61 g (50.0 mmol) 1-octene, 100.0 μL (1.00 μmol, 0.01 M in toluene) ethylene-bis-indenyl zirconium dichloride (Strem Chemical, Newburyport Mass.) followed by 0.58 ml (1.00 mmol, 1.7 M in toluene) methylalumoxane (Albemarle, Baton Rouge La.). The open end of the pouch was immediately heat sealed and the temperature of the contents of the pouch was monitored and recorded as a function of time. Table 4, below, reports time versus temperature data recorded for these trials.

TABLE 4
Time vs Temperature

| Time | Temp |
|---|---|
| (sec) | (° C.) |
| 0.0 | 29.7 |
| 10.0 | 30.0 |
| 20.0 | 30.3 |
| 30.0 | 30.6 |
| 40.0 | 31.1 |
| 50.0 | 31.3 |
| 60.0 | 31.6 |
| 70.0 | 32.1 |
| 80.0 | 32.1 |
| 90.0 | 32.8 |
| 100.0 | 33.1 |
| 110.0 | 33.4 |
| 120.0 | 33.7 |
| 130.0 | 34.0 |
| 140.0 | 34.3 |
| 150.0 | 34.4 |
| 160.0 | 35.0 |
| 170.0 | 35.5 |
| 180.0 | 35.8 |
| 210.0 | 36.7 |
| 240.0 | 37.5 |
| 270.0 | 38.8 |
| 300.0 | 40.0 |
| 330.0 | 41.8 |
| 360.0 | 42.8 |
| 390.0 | 44.2 |
| 420.0 | 45.6 |
| 450.0 | 47.0 |
| 480.0 | 47.8 |
| 510.0 | 49.0 |
| 540.0 | 50.4 |
| 570.0 | 51.1 |
| 600.0 | 52.7 |
| 630.0 | 54.2 |
| 660.0 | 55.9 |
| 690.0 | 59.0 |
| 720.0 | 61.2 |
| 750.0 | 62.2 |
| 780.0 | 66.1 |
| 810.0 | 69.5 |
| 840.0 | 73.4 |
| 870.0 | 77.7 |
| 900.0 | 82.2 |
| 930.0 | 86.9 |
| 960.0 | 91.9 |
| 990.0 | 92.8 |
| 1020.0 | 93.5 |
| 1050.0 | 97.2 |
| 1080.0 | 96.2 |
| 1110.0 | 87.5 |
| 1140.0 | 79.5 |
| 1170.0 | 77.7 |
| 1200.0 | 76.2 |
| 1230.0 | 79.0 |
| 1260.0 | 75.9 |
| 1290.0 | 74.1 |
| 1320.0 | 72.6 |
| 1350.0 | 71.3 |
| 1380.0 | 69.1 |
| 1410.0 | 63.3 |
| 1440.0 | 61.2 |
| 1470.0 | 59.2 |
| 1500.0 | 57.4 |

The data of Table 4 show that the temperature of the contents of the pouch increased with increased reaction time, and then decreased upon completion of the reaction. This example demonstrated real time, reversible, continuous monitoring.

### Example 5

A QCM instrument utilizing a quartz crystal (SC-501-1), probe (TPS-550) and monitor (PM-710, Maxtec, Santa Fe Springs Calif.) was sealed to one of the side walls of a piece of polyethylene bag tubing (as in Example 1) that measured 10 cm by 4.5 cm by 0.15 mm thick by placing the crystal retaining ring inside the polyethylene tubing and screwing it directly onto the probe which was placed outside the tubing. The piece of tubing covering the crystal was then carefully cut away using a scalpel. One end of the tubing was then heat sealed closed and 30.0 g of a solution of 99.8% 2-EHA and 0.2% Esacure KB1 photoinitiator (Sartomer, West Chester Pa.) was added. The solution was then stripped with N₂ for 20 minutes using an 18 gauge needle that was placed in the open end of the pouch and into the solution. The needle was removed and the open end of the tubing was quickly heat sealed closed. The QCM was attached to a frequency monitor and the pouch was exposed to an UV light source. The QCM resonant frequency was measured at various time intervals and is shown in Table 5.

TABLE 5
Frequency vs Time

| Time | Frequency |
|---|---|
| (sec) | (Hz) |
| 0 | 4.9978998 |
| 10 | 4.9978988 |
| 20 | 4.9978980 |
| 30 | 4.9978910 |
| 40 | 4.9978946 |
| 50 | 4.9978962 |
| 113 | 4.9983741 |
| 180 | 4.9983668 |
| 190 | 4.9892747 |
| 205 | 4.9889022 |
| 200 | 4.9887358 |
| 205 | 4.9887351 |
| 210 | 4.9887346 |
| 220 | 4.9887332 |
| 225 | 4.9887331 |
| 230 | 4.9887330 |
| 240 | 4.9887280 |
| 245 | 4.9887270 |
| 250 | 4.9887260 |
| 254 | 4.9887250 |
| 270 | 4.9887240 |
| 301 | 4.9887230 |
| 304 | 4.9887220 |
| 308 | 4.9887210 |
| 311 | 4.9887200 |
| 315 | 4.9887190 |
| 324 | 4.9887160 |
| 329 | 4.9887140 |
| 334 | 4.9887120 |
| 339 | 4.9887100 |
| 348 | 4.9887060 |
| 356 | 4.9887020 |
| 400 | 4.9887000 |
| 407 | 4.9886960 |
| 414 | 4.9886920 |
| 417 | 4.9886900 |
| 425 | 4.9886850 |
| 432 | 4.9886800 |
| 439 | 4.9886750 |
| 444 | 4.9886700 |
| 451 | 4.9886650 |
| 458 | 4.9886600 |
| 503 | 4.9886550 |
| 509 | 4.9886500 |
| 515 | 4.9886450 |
| 520 | 4.9886400 |
| 526 | 4.9886350 |
| 531 | 4.9886300 |
| 536 | 4.9886250 |
| 541 | 4.9886200 |
| 545 | 4.9886150 |
| 550 | 4.9886100 |
| 554 | 4.9886050 |
| 558 | 4.9886000 |
| 607 | 4.9885900 |
| 613 | 4.9885800 |
| 619 | 4.9885700 |

The data of Table 5 show that after an initial increase due to decreased density from the initial heat of polymerization, the frequency of the crystal decreased as the conversion of monomer to polymer increased, showing that the frequency was continuously monitored and was continuously responsive. Example 6

Into one of the open ends of a 10 cm piece of polyethylene bag tubing (4.5 cm diameter×0.5 mm thick) as in Example 1 was placed a diffuse reflectance probe (Catalog number R200-REF-VIS/NIR, Ocean Optics, Dunedin Fla.). The end of the tubing was then heat sealed close to the probe using an impulse heat sealer and the opening around the probe was sealed using 82518 RTV silicone rubber sealant (Loctite, Rocky Hill Conn.). To the open end of the tubing was added 3.2 g (30 mmol) 2-isopropylaniline, 2.18 g (15 mmol aqueous 40%) glyoxal, 30 ml ethanol and 0.05 g formic acid. The open end of the tubing was then heat sealed closed and the pouch was placed in a darkened container to limit stray light. A light source (LS-1 tungsten halogen lamp, Ocean Optics, Dunedin Fla.) was attached to the excitation end of the diffuse reflectance probe, and a spectrometer (SD2000, 100 micrometer slit, 600 lines/mm, Ocean Optics, Dunedin Fla.) was attached to the measuring end of the diffuse reflectance probe. The visible light transmission spectra of the mixture was monitored at various times over a period of five hours, as the reaction between the contents of the pouch proceeded. Data in the form of the value of the source corrected relative transmission at 619 nm was recorded with a computer, and is shown in tabular form in Table 6.

TABLE 6
Optical Transmission (counts at 619 nm) vs Time

| Time | |
|---|---|
| (min) | Counts |
| 0 | 153 |
| 4 | 140 |
| 8 | 129 |
| 12 | 125 |
| 16 | 122 |
| 20 | 117 |
| 24 | 117 |
| 28 | 116 |
| 32 | 117 |
| 36 | 116 |
| 40 | 116 |
| 44 | 112 |
| 48 | 112 |
| 52 | 108 |
| 56 | 108 |
| 60 | 106 |
| 64 | 106 |
| 68 | 103 |
| 72 | 100 |
| 76 | 100 |
| 80 | 98 |
| 84 | 92 |
| 88 | 100 |
| 92 | 96 |
| 96 | 97 |
| 100 | 92 |
| 104 | 94 |
| 108 | 91 |
| 112 | 87 |
| 116 | 88 |
| 120 | 87 |
| 124 | 86 |
| 128 | 84 |
| 132 | 84 |
| 136 | 80 |
| 140 | 83 |
| 144 | 79 |
| 148 | 79 |
| 152 | 78 |
| 156 | 73 |
| 160 | 74 |
| 164 | 74 |
| 168 | 73 |
| 172 | 68 |
| 176 | 66 |
| 180 | 65 |
| 184 | 66 |
| 188 | 67 |
| 192 | 62 |
| 196 | 63 |
| 200 | 63 |
| 204 | 60 |
| 208 | 62 |
| 212 | 61 |
| 216 | 58 |
| 220 | 61 |
| 224 | 57 |
| 228 | 58 |
| 232 | 62 |
| 236 | 56 |
| 240 | 60 |
| 244 | 60 |
| 248 | 61 |
| 252 | 61 |
| 256 | 59 |
| 260 | 59 |
| 264 | 59 |
| 268 | 60 |
| 272 | 57 |
| 276 | 59 |
| 280 | 57 |
| 284 | 59 |
| 288 | 60 |
| 292 | 59 |
| 296 | 60 |

The data of Table 6 show that over the course of the reaction, the relative value of optical transmission started at a maximum, and then diminished as the reaction proceeded until it reached an equilibrium state, demonstrating that the optical properties of the reaction inside the bag were continuously monitored and were continuously responsive. Example 7

An electric buzzer (70 dB PC Piezo Model 273-074, Radio Shack, Fort Worth Tex.) with a central Frequency of 5 kHz was connected to a 9 volt battery and a switch. This assembly was then placed inside a section of polyethylene bag tubing (4.5 cm diameter×0.5 mm thick, catalog no. 2062T23 McMaster Carr, Chicago Ill.), which was then heat sealed. This device was placed inside another piece of the same polyethylene bag tubing, one end of which was heat-sealed. The outer tubing was then filled with 100 g of 99.8% 2-EHA and 0.2% Esacure KB-1 photoinitiator, degassed with flowing nitrogen for five minutes, and then the remaining end was sealed.

The buzzer was turned on and the sample subjected to 5 minutes of irradiation with a 365 nm 8W UV source (UVP, Upland Calif.) in 30 second bursts. After each burst, the UV source was turned off and 10 seconds of the audible signal from the buzzer transduced through a microphone (D660S, AKG Acoustics, Nashville Tenn.) held two inches away from the sample, through a mixer (Eurorack model MX 802A-ULN, Behringer, Edmonds Wash.), and digitized into a laptop computer using Microsoft Sound Recorder v5.0. A fast Fourier transform was performed on the data. The maximum output frequency of the buzzer vs. time is reported in Table 7 below.

TABLE 7
Frequency vs Time

| Time (sec) | Frequency (Hz) |
|---|---|
| 0.5 | 4962.37 |
| 1.0 | 4964.335 |
| 1.5 | 4954.778 |
| 2.0 | 4179.11 |
| 2.5 | 3927.506 |
| 3.0 | 3867.99 |
| 3.5 | 4008.637 |
| 4.0 | 4035.396 |

The data show a transition from the free-flowing liquid to the rubbery polymeric state. This demonstrated use of a sensor comprising a free-floating responsive element which communicated remotely with an external processing element, and returned real-time information regarding the properties of materials in the pouch.

Various modifications and alterations of this invention will become apparent to those skilled in the art without departing from the scope and intent of this invention, and it should be understood that this invention is not to be unduly limited to the illustrative embodiments set forth herein.

It is claimed:

1. A reaction device comprising a fluid-impervious, flexible, pouch and an analytical sensor for real time, in situ, reversible measurement of properties of materials within the pouch.

2. The reaction device according to claim 1 wherein said sensor is integrally connected to said pouch.

3. The reaction device according to claim 1 wherein said sensor is within said pouch and is free of integral connection to said pouch.

4. The reaction device according to claim 1 wherein said analytical sensor comprises a responsive element, a processing element, and a means for transmitting information between said responsive element and said processing element.

5. The reaction device according to claim 4 wherein said responsive element is part of the body of the pouch.

6. The reaction device according to claim 5 wherein said responsive element is attached to the wall of the pouch.

7. The reaction device according to claim 5 wherein said responsive element is free of attachment to the pouch.

8. The reaction device according to claim 4 wherein said responsive element is sealed in an edge of the pouch.

9. The reaction device according to claim 4 wherein said responsive element is addressed by one or more electrodes located outside the pouch.

10. The reaction device according to claim 4 wherein said responsive element is addressed remotely.

11. The reaction device according to claim 4 wherein said transmitting means comprises one or both of electrical and optical elements.

12. The reaction device according to claim 4 wherein said transmitting means comprises one or both of mechanical and radiation elements.

13. The reaction device according to claim 12 wherein said radiation element provides radiation selected from the group consisting of acoustic waves, actinic radiation, nuclear radiation, and magnetism.

14. The reaction device according to claim 1 wherein said pouch further comprises one or more of reaction component(s), intermediate(s), and reaction product(s).

15. The reaction device according to claim 14 wherein said analytical sensor is responsive to selected material properties of one or more of reaction component(s), intermediates, and reaction product(s) in the pouch.

16. The reaction device according to claim 15 wherein said analytical sensor monitors a physical property.

17. The reaction device according to claim 15 wherein said analytical sensor monitors a chemical property.

18. The reaction device according to claim 17 wherein said chemical property monitored is polymer cure.

19. The reaction device according to claim 15 wherein said analytical sensor monitors a biological property.

20. The reaction device according to claim 1 wherein said analytical sensor is disposable.

21. The reaction device according to claim 4 wherein said responsive element is selected from the group consisting of thermocouples, interdigitated transducers (IDTs), and acoustic sensors (SAWS, QCMs).

22. The reaction device according to claim 4 wherein said responsive element of said analytical sensor is flexible.

23. The reaction device according to claim 4 wherein said responsive element comprises a flexible polymer film having metal circuit patterns deposited thereon.

24. The reaction device according to claim 1 wherein said pouch is comprised of a thermoplastic film.

25. A method for monitoring changes in material properties of contents in a reaction device comprising the steps of:
a) providing a reaction device comprising a flexible, sealed, fluid-impervious pouch including one or more reaction components, the reaction device also comprising an analytical sensor operating under a measurement protocol for real time, in situ, reversible measurement of properties of materials within the pouch, the analytical sensor comprising a responsive element for converting chemical or physical information (output) into electrical or electromagnetic signals, a processing element inside or on the pouch for converting electrical or electromagnetic signals into usable information, and a means for transmitting information between said responsive element and said processing element,
b) exposing the pouch to a controlled environment to cause the reaction components to interact to form one or more of reaction blends, products, and formulations, and
c) causing the responsive element and the processing element of the sensor to monitor changes in material properties occurring within the pouch, and
d) optionally, using the processing information to modify one or both of the controlled environment and the measurement protocol.

26. The method according to claim 25 wherein said pouch is sealed.

27. The method according to claim 25 wherein said pouch is self-supported.
